(19) United States
(12) Patent Application Publication
(10) Pub. No.: US 2015/0045619 A1
(43) Pub. Date: Feb. 12, 2015

(54) SYSTEM AND METHOD FOR MOSAICING ENDOSCOPE IMAGES USING WIDE ANGLE VIEW ENDOSCOPE

(71) Applicant: Chang Bing Show Chwan Memorial Hospital, Changhua County (TW)

(72) Inventors: Atul Kumar, Changhua County (TW); Ching-Jen Wu, New Taipei City (TW); Kai-Che Liu, Kaohsiung City (TW)

(73) Assignee: Chang Bing Show Chwan Memorial Hospital, Changhua County (TW)

(21) Appl. No.: 13/963,478

(22) Filed: Aug. 9, 2013

(51) Int. Cl. A61B 1/00; A61B 1/04

(52) U.S. Cl. CPC A61B 1/00009; A61B 1/042; A61B 1/00096; A61B 1/00174; A61B 1/00045
USPC 600/109

(57) ABSTRACT

A system for mosaicing endoscope images has an endoscope, a computer and a monitor. The endoscope has a relatively wide viewing angle such as 30 degrees to capture video images of a target area. The computer determines feature points on the captured video frames, aligns the video images in a common reference frame and displays them as one mosaiced image while dynamically extending the scene synchronous with the movement of the endoscope. The system creates a distortion free near real-time panorama of the endoscope scene using the wide angle view endoscope, and is useful for physicians while performing endoscopic procedures since the field of view can be extended to provide a better visual-spatial orientation.

### BACKGROUND OF THE INVENTION

1. Field of the Invention

The present invention relates to a system and a method for mosaicing endoscope images, and more particularly to a system and a method for a real-time mosaicing of endoscope images using a wide angle view endoscope.

2. Description of the Prior Art

Minimally invasive surgery (MIS) is an indispensable component of the current surgical procedures. The small size of incision and the shorter duration of hospital stay make MIS very attractive to the patients. MIS has gone through various advancements and is widely applied to different areas of surgery such as abdomen, pelvis and paranasal sinuses. One of the limitations of the MIS technique is the narrow field of view through the endoscope which impedes the visual-spatial orientation of the surgeon. A broader field of view is needed in getting the correct spatial orientation of the visual display space.

Creating a panoramic image based on the captured endoscopic images is one of the solutions for increasing the field of view. Mosaicing is a method of creating a panoramic image by stitching many small images together. Several studies have reported the application of the mosaicing techniques to create a panoramic view of organs such as a bladder, retina, placenta and sinus in an invasive surgery. All of these systems use feature extraction and tracking of those features in the video frames to create a panoramic view.

However, currently no endoscope mosaicing system uses wide angle view endoscope device for dynamically extending the field of the endoscope view, i.e. near real-time mosaicing of the endoscope images.

To overcome the shortcomings of the endoscopes, the present invention provides a system and a method for a near real-time mosaicing of endoscope images using a wide angle view endoscope to mitigate or obviate the aforementioned problems.

### SUMMARY OF THE INVENTION

The main objective of the present invention is to develop a system which would provide an extended dynamic field view during endoscopic procedures. The system uses an endoscope with a wide viewing angle to capture video images. The wide angle endoscope is used for the extended view of the scene and creating a mosaic view with such captured images.

The system for mosaicing endoscope images comprises the following components:

an endoscope having a body, an image channel extending from the body, a wide angle lens mounted to the tip of the image channel, and a camera in the body for capturing consecutive video images of a target area via the wide angle lens;

a computer connecting to and communicating with the endoscope for processing the captured consecutive video images received to correct the distortion due to the wide angle lens and to generate a mosaiced image; and

a monitor connecting to the computer for displaying the mosaiced image received from the computer.

In accordance with the present invention, the system uses the wide angle view endoscope to capture images. The wide angle view endoscope is able to capture a relative broader view of the scene compared to the conventional endoscopy. Therefore, the present system can create a larger view mosaic of the scene in near real-time.

Other objectives, advantages and novel features of the invention will become more apparent from the following detailed description when taken in conjunction with the accompanying drawings.

### BRIEF DESCRIPTION OF THE DRAWINGS

FIG. 1 is a block diagram of a system for mosaicing endoscope images in accordance with the present invention;

FIG. 2 is a flow chart of a method for mosaicing endoscope images in accordance with the present invention;

FIGS. 3A and 3B show two consecutive images captured by the endoscope of the present invention; and

FIG. 3C shows a panoramic view generated from the images of FIGS. 3A and 3B.

### DETAILED DESCRIPTION OF THE PREFERRED EMBODIMENTS

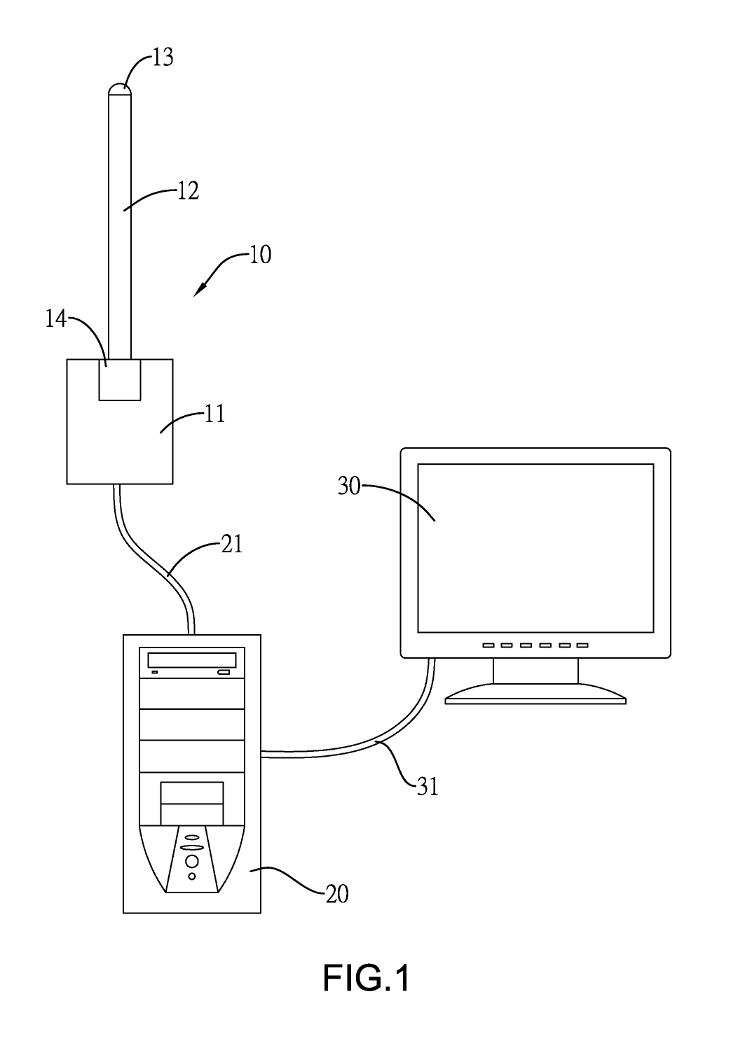

With reference to FIG. 1, a system for mosaicing endoscope images in accordance with a preferred embodiment of the present invention comprises an endoscope 10, a computer 20, and a monitor 30.

The endoscope 10 has a body 11, an imaging channel 12 extending from the body 11, a wide angle lens 13 is mounted to a tip of the imaging channel 12. The endoscope 10 electronically connects to and communicates with the computer 20 via a wired communication such as a cable 21 or a wireless communication. In this embodiment, the viewing angle of the wide angle lens 13 is 30 degrees.

The body 11 contains a camera 14 or a connection to a camera and a light source or a connection to a light source. The camera 14 may comprise, but is not limited to monochrome imagers, single CCD cameras, multi-CCD cameras. The endoscope camera transmits captured images to the computer 20 via the cable 21.

The computer 20 has the hardware and software to correct the wide angle lens distortion and then create a mosaiced image of the captured video images from the camera. The mosaiced image is displayed on the monitor 30 connected to the computer via a signal cable 31.

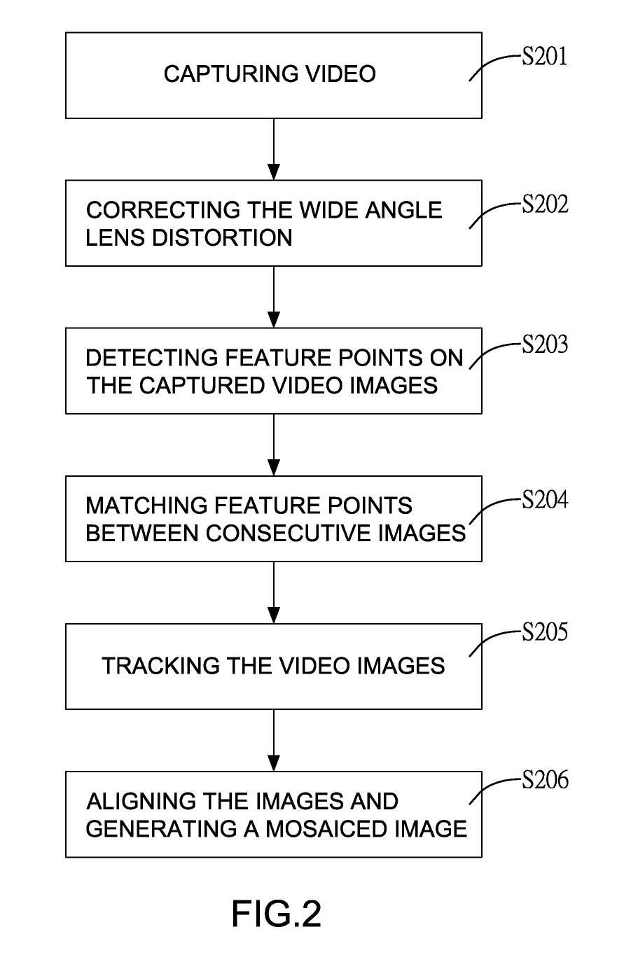

With reference to FIG. 2, the method of the present invention is executed by the system described above and comprises the steps of: capturing video (S201), correcting the wide angle lens distortion (S202), detecting feature points on the captured video images (S203), matching feature points between consecutive images (S204), tracking the video images (S205), aligning the images and generating a mosaiced image (S206).

In the step of capturing video (S201), the Karl Storz 26003 BA product may be used in the invention as the endoscope 10 to capture consecutive video images with the resolution of 720×480 pixels and frame rate (frame frequency) of upto 30 per second. The captured images are acquired at a proper frame rate chosen so that the motion occurring between the images is sufficiently small for the large number of feature points between overlapped images. The captured consecutive video images are transmitted to and processed by the computer 20 as described in the steps S202-S206. Preferably, the captured consecutive video images will be converted to a digital format for processing. If the captured video images are color images, they will be converted to grayscale images in advance for reducing processing time.

In the step of the wide angle distortion correction (S202), a fully automatic Hough-based calibration method and calibration results are applied to achieve real-time distortion correction, and a graphics processing unit (GPU) to interpolate image in parallel is used. This method of the correction of the distortion due to wide angle camera was described in detail in the article “Wide-angle distortion correction by Hough transform and gradient estimation” published in the proceedings of Visual Communications and Image Processing (VCIP), 2011 IEEE.

For the method, a formula of wide-angle lens distortion and focal length was derived, and a relationship between image boundary and focal length was established. It can estimate focal length on-line and achieve real-time distortion correction.

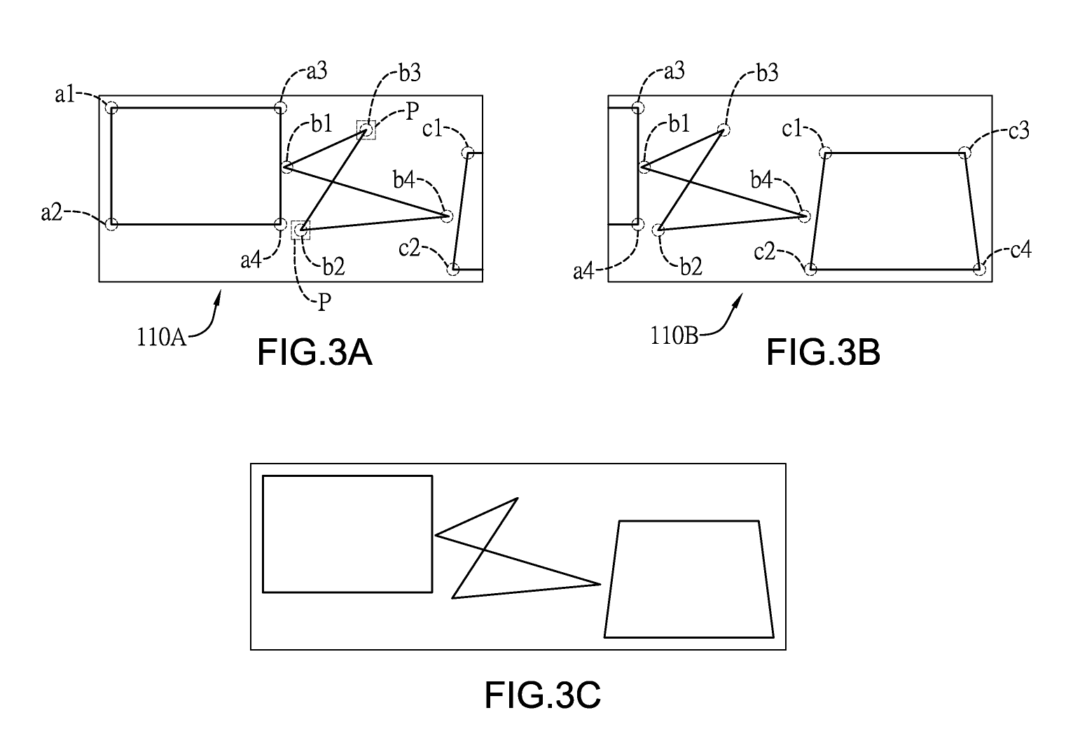

In the step of detecting feature points on the captured video images (S203), a speeded-up robust feature (SURF) detection technique is applied for detecting feature points a1-a4, b1-b4, c1-c4 on the captured video images 110A, 110B as shown in FIGS. 3A and 3B. The detection technique is a robust local feature point detector, presented by H. Bay et. al. published in Computer Vision-ECCV 2006 with the title of “Surf: Speeded up robust features”. The feature point detector is based on the Hessian matrix and the convolution of the Gaussian second order derivative with the input image. Thresholding and non-maximum suppression on the pixel intensity are applied in its 3×3×3 neighborhood to localize interest points over different scales of images.

In the step of matching feature points between consecutive video images (S204), each detected feature point a1-a4, b1-b4, c1-c4 is assigned a feature descriptor, using binary string feature point descriptor called as BRIEF described in research paper by M. Calonder etal. published in “Brief: Binary robust independent elementary features” in the journal of Computer Vision ECCV 2010. In the BRIEF method, a patch (P) of size 48 is created and smoothed around each of the detected feature points a1-a4, b1-b4, c1-c4, and multiple pairs of pixels (x, y) inside each patch are selected randomly. A binary vector based upon comparison of the intensities (I) of the pixels “x” and “y” is created such that the BRIEF descriptor is 1 if I(x)>I(y) otherwise the BRIEF descriptor is 0. For matching the descriptor, a nearest neighbor search is done in the descriptor space using Hamming distance measurements. A threshold of 5 distances is used for matching.

In the step of tracking the video images (S205), using the matched feature points a global transformation, called homography, for images is estimated. The homography is described by an affine transformation with six degrees of freedom and is estimated using Random Sample Consensus (RANSAC) fitting model described by Li et al. in “Computing homography with RANSAC algorithm: a novel method of registration”, in the proceedings of SPIE. This is an iterative process in which three sets of points among the matched points are selected randomly and a homography is estimated using only those points which satisfied the threshold of displacement. A reference coordinate system is made by the first video frame F0, and the subsequent images Fi are transformed by the global homography matrix. The local transformation between consecutive images is used to derive the global transformation.

In the step of aligning images and generating mosaiced image (S206), all images are brought to a single reference frame and stitched together based upon the tracked feature points to form a mosaiced image, i.e. a panoramic view as shown in FIG. 3C. The RGB values of the overlapped parts between any two images is averaged to reduce the effect resulted from difference in the color and intensity displayed in the final mosaiced image. As the endoscope 10 moves, the computer 20 conducts the foregoing steps in near real-time to update the mosaiced image.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A system for mosaicing endoscope images comprising:
an endoscope having a body, an image channel extending from the body, a wide angle lens mounted to the tip of the image channel, and a camera in the body for capturing consecutive video images of a target area via the wide angle lens;
a computer connecting to and communicating with the endoscope for processing the captured consecutive video images received to generate an mosaiced image; and
a monitor connecting to the computer for displaying the mosaiced image received from the computer.

2. The system as claimed in claim 1, wherein a viewing angle of the wide angle lens is 30 degrees.

3. The system as claimed in claim 1, wherein the computer converts the captured consecutive video images to digital format.

4. The system as claimed in claim 1, wherein the computer corrects distortions in the video images due to the wide angle lens of the endoscope.

5. The system as claimed in claim 1, wherein the computer continuously updates the mosaiced image synchronous with the movement of the endoscope.

6. A method for mosaicing endoscope images comprising the steps of:
using an endoscope with a wide viewing angle to capture consecutive video images of a target area;
transmitting the consecutive video images to a computer for image processing, wherein the image processing further has the steps of:
using a speeded-up robust feature (SURF) detection technique to detect feature points on the consecutive video images;
matching feature points between consecutive video images;
tracking the video images by using a homography global transformation; and
aligning the video images based on the tracked feature points and generating the mosaiced image; and
updating the mosaiced image in synchrony with a movement of the endoscope.

7. The method as claimed in claim 6, wherein a viewing angle of the endoscope is 30 degrees.

8. The method as claimed in claim 7, wherein the endoscope captures the consecutive video images with the resolution of 720×480 pixels and frame rate (frame frequency) of up to 30 per second.

9. The method as claimed in claim 7, wherein the image processing further has a step of converting the video images from color to grayscale before feature points detection.

10. The method as claimed in claim 7, wherein the computer performs the image processing in near real-time.
